(19) United States
(12) Plant Patent Application Publication
(10) Pub. No.: US 2002/0166153 P1
(43) Pub. Date: Nov. 7, 2002

(54) CORYDALIS FLEXUOSA PLANT NAMED 'GOLDEN PANDA'

(75) Inventor: Dan M. Heims, Portland, OR (US)

Correspondence Address: KLARQUIST SPARKMAN CAMPBELL LEIGH & WHINSTON, LLP, One World Trade Center, Suite 1600, 121 S.W. Salmon Street, Portland, OR, 97204, US

(73) Assignee: Reuben A. Hatch

(21) Appl. No.: 09/848,893

(22) Filed: May 3, 2001

(51) Int. Cl. A01H 5/00

(52) U.S. Cl. PLT/263

(57) ABSTRACT

A new and distinct form of Corydalis plant characterized by its distinctive yellow variegated leaves.

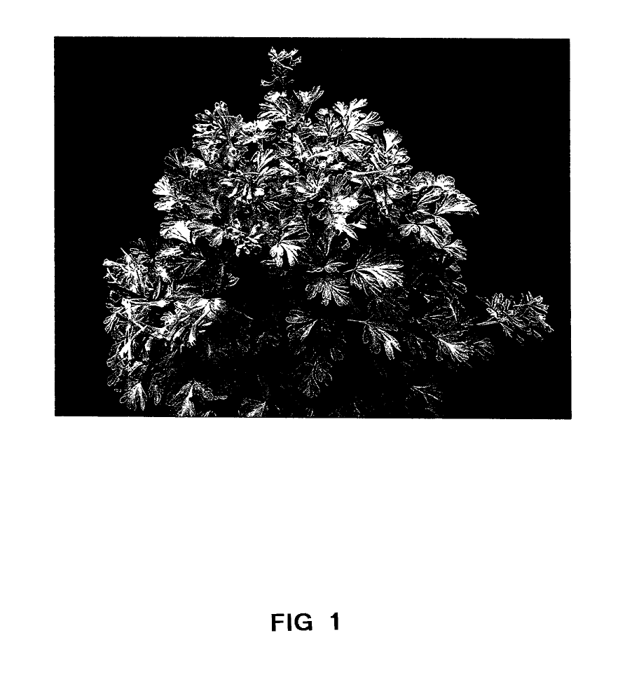

The present invention relates to a new and distinct hybrid of Corydalis, of the family Papaveraceae, which originated from a mutation in tissue culture from an unpatented Corydalis flexuosa ‘Blue Panda’.

This plant is characterized by the unique variegation of its foliage, which is patterned and colored with shades of yellow.

The new variety has been reproduced only by asexual propagation (division and micropropagation). Each of the progeny exhibits identical characteristics to the original plant. Asexual propagation by division and micropropagation as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The drawing shows a two-year-old Corydalis flexuosa ‘Golden Panda’ in bloom in early summer.

### DETAILED PLANT DESCRIPTION

The following is a detailed description of the new Corydalis flexuosa variety based on observations of two-year-old specimens grown in two-gallon containers in a cool greenhouse in Canby, Oreg. The color descriptions are all based on The Royal Horticultural Society Colour Chart.

Plant:

Form.—Clumping, herbaceous perennial, non-running habit.

Cold Hardiness.—USDA Zone 5.

Size.—to 20 cm tall and 30 cm wide.

Stem:

Type.—Rhizomatous, covered with fleshy scales.

Size.—About 0.7 cm wide.

Color.—Greyed Orange 174B

Leaf:

Type.—Compound, bitemate.

Arrangement.—basal.

Shape.—Ovate in outline.

Lobing/Division.—Each secondary leaflet is temately divided and each division is 2-3 lobed.

Venation.—Palmate.

Margins.—Deeply divided, each lobe is entire.

Apex.—Mucronulate.

Base.—Lateral leaflets cordate, terminal leaflets truncate to cuneate.

Size.—to 4 cm long and 3.25 cm wide.

Texture.—Glabrous.

Petiole length.—to 8 cm long.

Petiole texture.—Glabrous.

Petiole color.—Greyed Orange 174A-B to Greyed Red 182A.

Leaf color.—Variegated with leaflet centers irregularly Yellow 4D to 2C streaking to greener toward margins Yellow Green 144A to Yellow Green 146C and 147C. Winter and early spring leaves are Green 138A with streaks of Yellow Green 145A.

Inflorescence:

Type.—Raceme.

Number of Flowers.—to about 11 per raceme.

Peduncle.—Height—about 14 cm. Diameter—0.15 cm. Color—Greyed Yellow 161A-B. Texture—Glabrous.

Pedicel.—Length—to about 1.5 cm. Texture—Glabrous. Color—Greyed Red 179C-D.

Bloom period.—April to June in Canby, Oreg.

Flower Bud:

Size.—about 1.7 cm prior to opening.

Description.—Shaped like a young tadpole.

Color.—Blue 106A-B with White 155D on bottom side.

Flower:

Type.—Zygomorphic.

Shape.—Tubular. Upper petal with a nectary spur which curves up. Nectary spur extends back from pedicel for 0.6 cm.

Size.—About 2.5 cm long and 0.6 cm at tip where it is widest.

Color.—Blue 106A-B on upper petal and tip of lower petal. White 155D on lower petal and inner petals.

Petal count.—4.

Sepal #.—0.

Stamen number.—2.

Pistil.—About 1.1 cm long and white 155A.

Pollen.—Yellow 13C.

Filaments.—Flat, translucent, 0.4 cm long.

Fragrance.—Reminiscent of sweet violet.

Lastingness.—A raceme blooms for about 3 weeks on the plant.

Fruit:

Type.—Narrowly linear, pendent capsules.

Fertility.—Low.

Disease: None observed on plants grown commercially in Canby, Oreg.

### DESCRIPTION AND COMPARISON OF PARENT PLANT

Corydalis flexuosa ‘Blue Panda’ has pale blue-green, unmarked, fern-like leaves and sky-blue flowers with curved spurs. It is clump-forming with a non-running habit. Compared to Corydalis flexuosa ‘Blue Panda’, Corydalis flexuosa ‘Golden Panda’ is a stable variegated form with leaves which turn golden in the summer. Both plants have excellent form, vigor, and blooms.

I claim:

1. A new and distinct form of Corydalis plant substantially as shown and described, characterized by a distinct yellow leaf coloration and pattern.
